(19) United States
(12) Patent Application Publication
(10) Pub. No.: US 2004/0059257 A1
(43) Pub. Date: Mar. 25, 2004

(54) Deflectable guiding apparatus

(76) Inventor: Benny Gaber, Haifa (IL)

Correspondence Address: David Klein, Dekel Patent, Beit HaRof im Room 27, 18 Menuha VeNahala Street, Rehovot, 76209, IL

(21) Appl. No.: 10/450,446

(22) PCT Filed: Dec. 24, 2001
(86) PCT No.: PCT/IL01/01196

(30) Foreign Application Priority Data
Jan. 8, 2001 (IL) 140780

(51) Int. Cl. A61B 5/00

(52) U.S. Cl. 600/585

(57) ABSTRACT

Guiding apparatus including a generally hollow tube formed with a plurality of radial slits spaced axially from each other, the slits extending partially through a circumference of the tube, the tube being sufficiently flexible so as to bend upon application of a force that compresses at least one of the slits toward another of the slits.

### FIELD OF THE INVENTION

The present invention relates to guiding apparatus in general, and particularly to deflectable guiding apparatus for advancing catheters within body lumens, or deflectable catheters themselves, in procedures such as percutaneous transluminal coronary angioplasty (PTCA).

### BACKGROUND OF THE INVENTION

In order to facilitate the advancement of catheters through a body lumen (e.g., an artery), deflectable catheters have been developed. The simultaneous application of torque at the proximal end of a catheter and the ability to selectively deflect the distal tip of the catheter in a desired direction permits the physician or surgeon to adjust the direction of advancement of the distal end of the catheter, as well as to position the distal portion of the catheter during, for example, PTCA.

A surgeon may manipulate the proximal end of the catheter to guide the catheter through a patient's vasculature. The deflection of the distal tip is typically provided by a pull wire that is attached at the distal end of the catheter and extends to a control handle that controls the application of tension on the pull wire.

In PTCA catheter designs it is critically important to have sufficient flexibility in the catheter shaft so that when the catheter is advanced through a blood vessel the catheter may follow the inherent curvature of the vessel without puncturing the vessel wall.

In order to maneuver around turns and bends in the vasculature, the surgeon typically observes the catheter fluoroscopically, and selectively deflects the tip and rotates the proximal end of the catheter shaft. However, the ability to control the precise position of the distal end of the catheter depends on the fidelity of the catheter's transmission of the forces exerted at the proximal end to the distal tip.

Without high fidelity torque transmission, the surgeon is unable to control the catheter tip and at best only delays an operating procedure, and at worst may cause the distal tip of the catheter to cause trauma to a patient.

A typical example of a prior art steerable catheter is that found in U.S. Pat. No. 5,865,800 to Mirarchi et al., which describes an axially elongated steerable catheter. The catheter has a distal tip portion deflectable in response to a pull wire within the catheter. The catheter is capable of being torqued at its proximal portion to change the rotational orientation of the tip portion about the longitudinal axis. The catheter includes an elongated flexible outer hollow catheter body; a deflectable tip portion deflectable relative to the hollow catheter body; and a pull wire extending through the catheter to a region on the deflectable tip portion distal of the region about which the tip portion deflects. The pull wire is arranged to apply tension on the deflectable portion to produce tip deflection. An elongated wound wire coil extends through the hollow catheter body of the steerable catheter. The coil is constructed and arranged to enable the catheter body to withstand reactive compressive load without distortion during application of tension on the pull wire and to transmit torque along the longitudinal axis from the proximal portion to the distal tip portion of the catheter to enhance the fidelity of rotational positioning of the distal tip portion in response to change of rotational orientation of the proximal portion of the catheter.

A major requirement for guidewires and other guiding members, whether they be solid wire or tubular members, is that they have sufficient columnar strength to be pushed through a patient's vascular system or other body lumen without kinking. However, they must also be flexible enough to avoid damaging the blood vessel or other body lumen through which they are advanced. Efforts have been made to improve both the strength and flexibility of guiding members to make them more suitable for their intended uses, but these two properties are for the most part diametrically opposed to one another in that an increase in one usually involves a decrease in the other.

### SUMMARY OF THE INVENTION

The present invention seeks to provide improved deflectable guiding apparatus for advancing catheters within body lumens. The guiding apparatus of the invention employs a tube formed with a plurality of radial slits spaced axially from each other. The slits extend partially through a circumference of the tube. When a compressive force is applied to the slits, such as by pulling with a pull wire attached to a distal portion of the tube, the tube buckles and bends towards the side of the slits. The tube buckles only upon application of the pull wire force, and has the requisite strength and flexibility for medical guiding members. The invention enables a surgeon to easily introduce, deflect and navigate the guiding apparatus through lumens and bifurcations. The surgeon can easily turn the guiding apparatus so that the guiding apparatus bends towards the intended direction when navigating through vasculature. The guiding apparatus may be used as a guiding support for catheters or may be part of the catheter itself.

There is thus provided in accordance with a preferred embodiment of the invention guiding apparatus including a generally hollow tube formed with a plurality of radial slits spaced axially from each other, the slits extending partially through a circumference of the tube, the tube being sufficiently flexible so as to bend upon application of a force that compresses at least one of the slits towards another of the slits.

In accordance with a preferred embodiment of the invention the radial slits are formed on one side of the tube, and the tube is adapted to bend towards the side of the slits upon application of a force that compresses at least one of the slits towards another of the slits.

Further in accordance with a preferred embodiment of the invention at least two of the radial slits are spaced generally equally from each other. Alternatively or additionally, at least two of the radial slits may be spaced unequally from each other.

In accordance with a preferred embodiment of the invention the radial slits extend at least half way through the circumference of the tube.

In accordance with an alternative embodiment of the invention, the radial slits are formed as grooves in a wall of the tube, and do not pass through a thickness of the wall of the tube.

In accordance with yet another embodiment of the invention, a wall of the tube does not have a uniform thickness. Preferably the thickness of the wall on a side of the slits is thinner than the side opposite the slits.

Further in accordance with a preferred embodiment of the invention a pull wire is attached to a distal portion of the tube. Preferably the pull wire is attached to the tube distally of all the slits.

Still further in accordance with a preferred embodiment of the invention the radial slits are formed on one side of the tube and the pull wire is attached to the tube on the side of the slits.

In accordance with a preferred embodiment of the invention a distal tip of the tube is softer than the rest of the tube. Alternatively, a tip member is attached to a distal end of the tube, which is softer than the rest of the tube.

There is also provided in accordance with a preferred embodiment of the invention guiding apparatus including a generally hollow tube that has an unevenly thick wall at a distal portion thereof, wherein the thickness of the wall on one side of the tube is thinner than an opposite side of the tube, the tube being sufficiently flexible so as to bend towards the thinner side of the tube upon application of a proximally-directed axial force upon the tube.

### BRIEF DESCRIPTION OF THE DRAWINGS

The present invention will be understood and appreciated more fully from the following detailed description taken in conjunction with the drawings in which:

FIGS. 1, 2 and 3 are simplified, partially sectional, side-view and bottom-view illustrations, respectively, of guiding apparatus, constructed and operative in accordance with a preferred embodiment of the invention;

FIG. 4A is a simplified sectional illustration of the guiding apparatus of FIGS. 1-3, taken along lines IV-IV in FIG. 1, wherein slits of the guiding apparatus extend at least half way through the circumference of a tube of the guiding apparatus;

FIG. 4B is a simplified sectional illustration of the guiding apparatus of FIGS. 1-3, taken along lines IV-IV in FIG. 1, showing an alternative construction wherein the slits do not pass through the thickness of the tube wall, but rather are formed as grooves in the outer side of tube;

FIG. 4C is a simplified sectional illustration of the guiding apparatus of FIGS. 1-3, taken along lines IV-IV in FIG. 1, showing yet another construction with slits and wherein the tube wall does not have a uniform thickness;

FIG. 4D is a simplified sectional illustration of the guiding apparatus of FIGS. 1-3, taken along lines IV-IV in FIG. 1, showing still another construction without slits and wherein the tube wall does not have a uniform thickness;

FIG. 5 is a simplified side-view illustration of a distal portion of the guiding apparatus of FIGS. 1-3 deflected by means of a pull wire slightly pulled proximally; and

FIG. 6 is a simplified side-view illustration of the distal portion of the guiding apparatus of FIGS. 1-3 deflected further by pulling the pull wire further proximally.

### DETAILED DESCRIPTION OF A PREFERRED EMBODIMENT

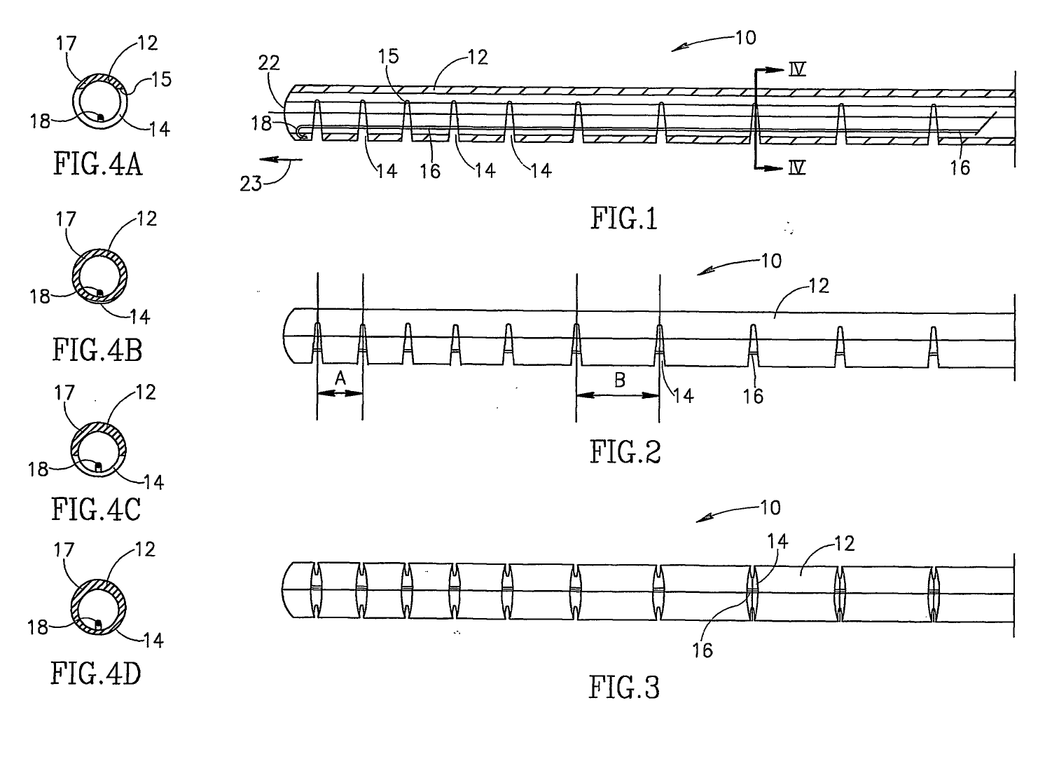

Reference is now made to FIG. 1, which illustrates guiding apparatus 10, constructed and operative in accordance with a preferred embodiment of the present invention. Guiding apparatus 10 may be used as a guiding support for catheters or may be part of the catheter itself.

Guiding apparatus 10 includes a generally hollow tube 12, preferably made of a flexible material, such as a resilient metal, plastic or composite material. An example of a suitable material is NITINOL, which is an acronym for Ni—Ti (nickel-titanium) Naval Ordnance Laboratory. NITINOL is an alloy that has shape memory and/or superelastic characteristics and is safe for use in medical devices.

A plurality of radial slits 14 are formed in tube 12, spaced axially from each other. Some or all of the slits 14 may be equally spaced from each other, or some or all of the slits 14 may be unequally spaced from each other, or a combination of both, as in the illustrated embodiment. (For example, some of the slits in the illustrated embodiment are separated by a distance A, while others are spaced by a distance B.) Slits 14 extend partially through a circumference of tube 12 up to a slit end 15. Slits 14 are preferably formed on one side of tube 12.

As seen in FIG. 2, and additionally in FIG. 4A, in one embodiment, slits 14 extend at least half way through the circumference of tube 12. In another embodiment, shown in FIG. 4B, slits 14 do not pass through the thickness of a wall 17 of tube 12, but rather are formed as grooves or partial bellows in the outer (or inner) side of tube 12. In this manner, tube 12 may be used to deliver fluids therethrough, and still bend and buckle.

In yet another embodiment, shown in FIG. 4C, the wall 17 of tube 12 does not have a uniform thickness. Rather, the thickness of wall 17 on the side of slits 14 is thinner than the side opposite slits 14. Such an embodiment may facilitate bending tube 12.

An alternative to the previous embodiment is shown in FIG. 4D. In this embodiment, the wall 17 of tube 12 does not have a uniform thickness, and there are no slits. Rather, tube 12 is generally smooth inside and outside, and the thickness of wall 17 on one side of tube 12 is thinner than the opposite side of tube 12. In such an embodiment, tube 12 bends towards its thinner side upon application of a proximally-directed axial force upon tube 12.

A pull wire 16 is preferably attached to a distal portion 18 of tube 12, most preferably distally of all of slits 14. As seen in FIG. 1, pull wire 16 is preferably attached to tube 12 on the same side of slits 14. Pull wire 16 extends through tube 12 and exits through a proximal end of tube 12. The proximal end of pull wire 16 may be manipulated by a surgeon as with any conventional pull wire.

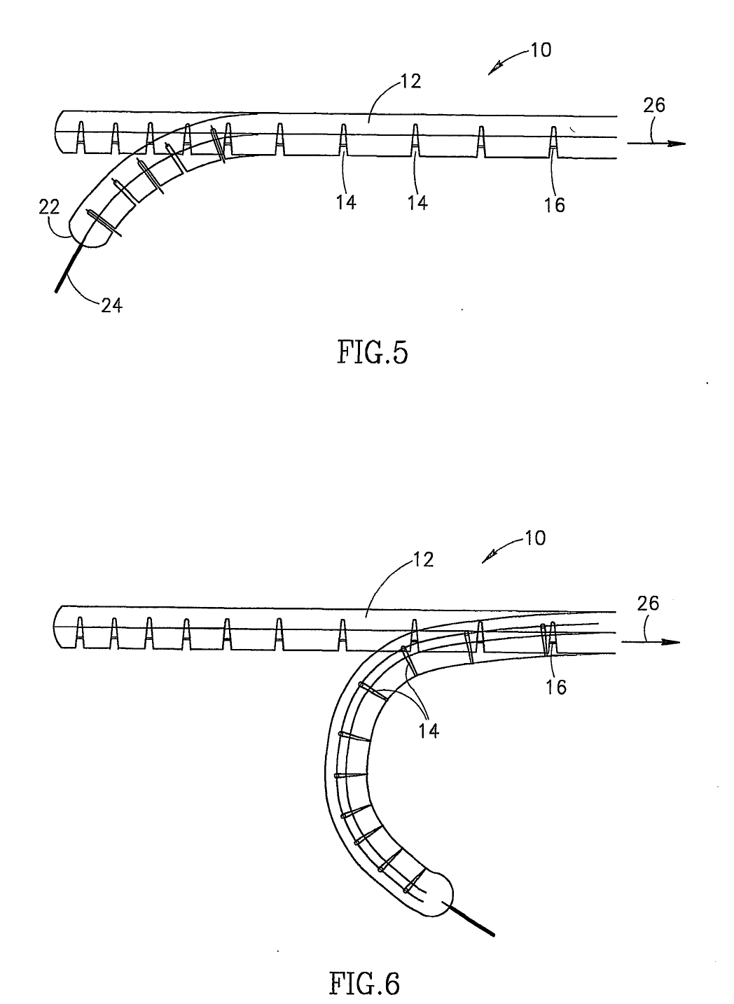

In order to facilitate movement of tube 12 through lumens and bifurcations, generally in the direction of an arrow 23 in FIG. 1, distal tip 22 of tube 12 may be made of a material softer than the rest of tube 12. Alternatively, a tip member 24 (shown in FIGS. 5 and 6) may be attached to the distal end of tube 12, the tip member 24 being softer than the rest of tube 12.

Reference is now made to FIGS. 5 and 6. By pulling pull wire proximally generally in the direction of an arrow 26, a compressive force is applied to slits 14. The compression of slits 14 towards each other causes the distal end of tube 12 to buckle and bend towards the side of slits 14. Tube 12 buckles basically in accordance with Euler's law of buckling (although the invention does not necessarily have to obey in accordance with this law). According to Euler's law, the axial buckling force on a slender member (like tube 12) is approximately equal to d⁴E/l², wherein d is a characteristic diameter, E is the modulus of elasticity of the material, and l is a characteristic length.

Positioning slits 14 on one side of tube 12 causes tube 12 to bend in that direction. The width of slits 14 permits the slits to be “pinched” as the tube bends. The curvature of the tube 12 is basically the sum of the bending of all the segments of tube material between the slits 14. As seen by comparing FIG. 5 with FIG. 6, the more pull wire 16 is pulled proximally, the more tube 12 bends and has a tighter bending radius. A surgeon can easily turn guiding apparatus 10 so that guiding apparatus 10 bends towards the intended direction when navigating through vasculature.

It will be appreciated by person skilled in the art, that the present invention is not limited by what has been particularly shown and described herein above. Rather the scope of the present invention is defined only by the claims that follow:

What is claimed is:

1. Guiding apparatus comprising:
a generally hollow tube formed with a plurality of radial slits spaced axially from each other, said slits extending partially through a circumference of said tube, said tube being sufficiently flexible so as to bend upon application of a force that compresses at least one of said slits towards another of said slits.

2. Guiding apparatus according to claim 1 wherein said radial slits are formed on one side of said tube, and said tube is adapted to bend towards the side of the slits upon application of a force that compresses at least one of said slits towards another of said slits.

3. Guiding apparatus according to claim 1 wherein at least two of said radial slits are spaced generally equally from each other.

4. Guiding apparatus according to claim 1 wherein at least two of said radial slits are spaced unequally from each other.

5. Guiding apparatus according to claim 1 wherein said radial slits extend at least half way through the circumference of said tube.

6. Guiding apparatus according to claim 1 wherein said radial slits are formed as grooves in a wall of said tube, and do not pass through a thickness of the wall of said tube.

7. Guiding apparatus according to claim 1 wherein a wall of said tube does not have a uniform thickness.

8. Guiding apparatus according to claim 7 wherein the thickness of said wall on a side of said slits is thinner than the side opposite said slits.

9. Guiding apparatus according to claim 1 and further comprising a pull wire attached to a distal portion of said tube.

10. Guiding apparatus according to claim 9 wherein said pull wire is attached to said tube distally of all said slits.

11. Guiding apparatus according to claim 9 wherein said radial slits are formed on one side of said tube and said pull wire is attached to said tube on the side of the slits.

12. Guiding apparatus according to claim 1 wherein a distal tip of said tube is softer than the rest of said tube.

13. Guiding apparatus according to claim 1 and further comprising a tip member attached to a distal end of said tube, which is softer than the rest of said tube.

14. Guiding apparatus comprising:
a generally hollow tube that has an unevenly thick wall at a distal portion thereof, wherein the thickness of the wall on one side of said tube is thinner than an opposite side of said tube, said tube being sufficiently flexible so as to bend towards the thinner side of said tube upon application of a proximally-directed axial force upon said tube.
